(19) United States
(12) Patent Application Publication
(10) Pub. No.: US 2005/0235993 A1
(43) Pub. Date: Oct. 27, 2005

(54) Ventilator, in particular CPAP device comprising an illumination device

(76) Inventors: Martin Baecke, Dessau (DE); Uwe Heimhalt, Kothen (DE); Ewald Anger, Eibelstadt (DE); Jurgen Reinstadtler, Wurzburg (DE)

Correspondence Address: KING & SCHICKLI, PLLC, 247 NORTH BROADWAY, LEXINGTON, KY, 40507, US

(21) Appl. No.: 11/132,690

(22) Filed: May 19, 2005

Related U.S. Application Data
(63) Continuation of application No. PCT/DE03/03593, filed on Oct. 29, 2003.

(30) Foreign Application Priority Data
Nov. 19, 2002 (DE) 102 53 934.0

(51) Int. Cl. A62B 7/00; A61M 16/00; F16K 31/02

(52) U.S. Cl. 128/204.18; 128/204.23

(57) ABSTRACT

The present invention relates to a respiratory apparatus, in particular a CPAP-apparatus. The apparatus comprises an illumination device for illuminating a display or operating members, and a controller connected with the illumination device for switching the illumination on and off. The apparatus moreover comprises a sensor likewise connected with the controller and outputting a signal to the controller which causes the controller to switch on the illumination.

### CROSS REFERENCE TO RELATED CO-PENDING APPLICATIONS

This application is a continuation of international application number PCT/DE03/003593 (publication number: WO 2004/045692 A1) filed on Oct. 29, 2003 and entitled VENTILATOR, IN PARTICULAR CPAP DEVICE COMPRISING AN ILLUMINATION DEVICE and claims the benefit of the above-mentioned international application and the corresponding German national patent application number 102 53 934.0-09 filed on Nov. 19, 2002 and entitled BEATMUNGSGERÄT, INSBESONDERE CPAP-GERÄT MIT EINER BELEUCHTUNGSEINRICHTUNG the contents of which are expressly incorporated herein by reference.

### FIELD OF THE INVENTION

The invention relates to a respiratory apparatus according to the preamble of claim 1, in particular to a CPAP-apparatus and a controller for switching an illumination device on and off.

### BACKGROUND OF THE INVENTION

“Clapping switches” are known from the prior art, which react on acoustic pulses such as clapping. They can comprise, for example, a plug for the mains supply and a corresponding socket. The clapping switch is supplied with electrical power via the plug and the mains supply. Upon initial clapping it connects its socket to the mains supply, after clapping a second time it disconnects the socket from the mains supply. If the plug of a lamp is plugged into the socket of the clapping switch light can be switched on and off by clapping.

Additionally known are lamps with movement detectors and light sensors. Such lamps are used, for example, to illuminate front door areas only when necessary. If the light sensor senses that it gets dark, the light is switched on if the movement detector also detects the movement of a person. A person not knowing the surroundings thus need not look for a possibly non-illuminated switch in the darkness.

Also the display of mobile telephones, e.g. the S35 of Siemens, is equipped with a display illumination and an illumination of the key legend. Upon pressing any key, the display and key illumination is switched on. If no key is pressed for 10 to 15 seconds, the display and key illumination is switched off again.

Known are moreover respiratory apparatus or respirators for the mechanical, artificial respiration for all forms of an oxygen deficiency state. They are, inter alia, applied for the long-time respiration. (Roche Medical Dictionary, 4^{th} Edition, published by Hoffmann-La Roche AG and Urban & Fischer, Munich, Stuttgart, Jena, Lübeck, Ulm).

Known are in particular apparatus for performing the CPAP therapy (continuous positive airway pressure). The CPAP therapy is described in Chest. Volume No. 110, pages 1077-1088, October 1996 and in Sleep, Volume No. 19, pages 184-188. A CPAP-apparatus applies, by means of a compressor and via a hose and a nose mask, a positive overpressure up to approximately 30 mbar to the respiratory tract of the patient. This positive pressure is to ensure that the upper respiratory tract remains fully opened during the whole night, so that no obstructive apneas will occur (DE 198 49 571 A1).

FIG. 1 shows a CPAP-apparatus 1 and a patient 19. The CPAP-apparatus comprises a housing 4, a respiratory hose 9 and a respiratory mask 18. For the generation of a positive pressure the housing 4 includes a turbine 8. The turbine is also designated as blower, blower unit, compressor, ventilator or fan. In this patent these terms are used synonymously. Furthermore, a pressure sensor 11 and optionally a flow sensor 16 are accommodated in the housing. In the mask, or in the proximity of the mask, one or several small holes 2 are provided which allow the formation of an air flow from the compressor to the holes 2 on the time average. This prevents the accumulation of CO₂ in the respiratory hose 9 and allows the supply of the patient with oxygen.

The speed of the turbine 8 is controlled by a microcontroller 5 such that the actual pressure measured with the pressure sensor 11 corresponds to a predetermined target pressure. The target pressure is typically preadjusted under the supervision of a medical practitioner and is called titration pressure. The flow sensor can, for example, be a sensor comprising a heating filament 17.

### SUMMARY OF THE INVENTION

According to an embodiment of the invention a respiratory apparatus is provided which comprises an illumination device, a controller and a sensor. The controller is connected with the illumination device for switching the illumination on and off. A sensor is connected to the controller and outputs a signal to the controller which causes the controller to switch on the illumination.

According to another embodiment of the invention a method for a respiratory apparatus is provided. The method comprises outputting a sensor signal from a sensor of the respiratory apparatus to a controller of the respiratory apparatus which causes the controller to switch on the illumination. Further, a light source is illuminated in response to the sensor signal.

It is an advantage of the use of a microphone for switching the illumination on that the patient need not even move in order to operate the respiratory apparatus.

With respect to switching the illumination on at loud ambient noise and with respect to switching the illumination off in a noiseless environment it is an advantage that this criterion frequently automatically switches the illumination on and off correctly. When the patient sleeps it is usually noiseless in the sleeping room. When the patient wakes up he or she makes noise by rustling with the blanket. At this time it is sensible that the illumination be switched on.

By the detection of amplitude variations in the ambient noise it can be detected whether someone speaks in the ambience of the respiratory apparatus. Thus, the patient may talk to the apparatus in the dark so as to switch on the illumination thereof.

It is advantageous with respect to an accelerometer that it senses the slight concussions caused, for example, by patient's groping for the respiratory apparatus.

An infrared sensor, such as a photodiode or a pyrosensor, is an inexpensive movement sensor which also works in the dark.

An advantage of a housing window pressing on an infrared sensor resides in that, when correctly selecting the infrared sensor, also movements of the housing and, thus, of the housing window result in a sensor signal. By this the infrared sensor not only detects movements of a hand in front of the respiratory apparatus, but also slight concussions of the apparatus caused by groping movements.

An advantage of a lens or lens system in front of the infrared sensor resides in that the solid angle may be defined more exactly due to the reaction of the infrared sensor on movements.

Also a contact sensor advantageously switches the illumination of the respiratory apparatus on when the patient gropes for the respiratory apparatus.

### BRIEF DESCRIPTION OF THE DRAWINGS

Preferred embodiments of the invention will hereinafter be explained in more detail with reference to the enclosed drawings, wherein like numerals represent like parts.

FIG. 1 shows a CPAP-apparatus according to the invention,

FIG. 2 shows a circuit for a contact sensor,

FIG. 3 shows a thermopile for the infrared detection with a corresponding circuitry, and

FIG. 4 shows the inventive installation of the infrared sensor.

### DETAILED DESCRIPTION OF THE INVENTION

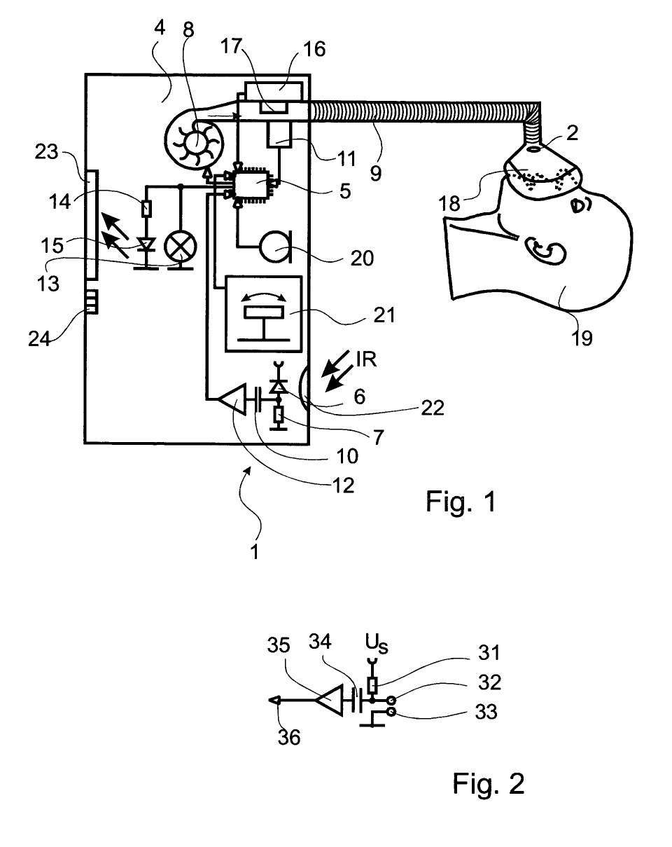

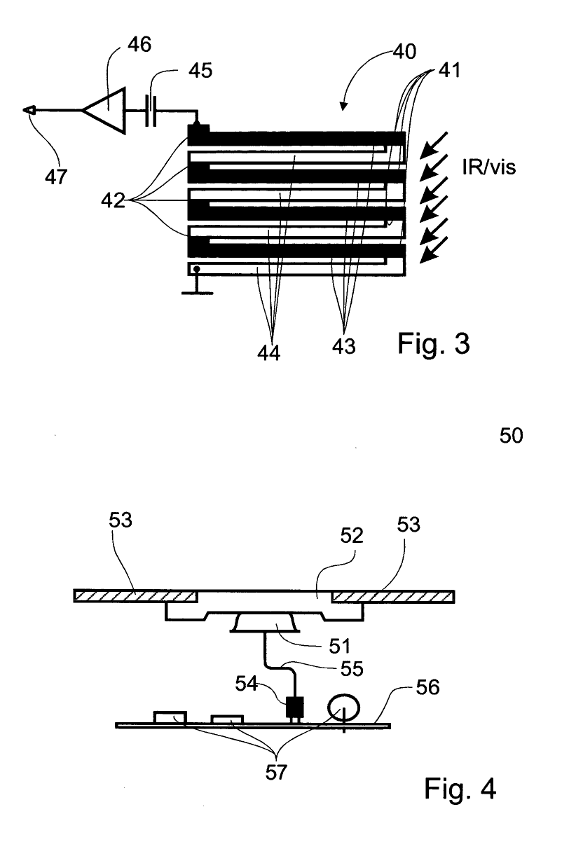

The CPAP-apparatus according to the invention shown in FIG. 1 moreover comprises a display 23, preferably an LCD (liquid crystal display) and/or at least partially transparent operating members 24. The display and the operating members can be illuminated by an illumination device or light source which may be one or more light emitting diode(s) 15 or one or more light bulb(s) 13. For a stable operation of light emitting diodes a current limiter, e.g. in the form of a resistor 14, is necessary.

Moreover, the CPAP-apparatus according to the invention comprises at least one sensor, which may be a microphone 20, an accelerometer 21, an infrared sensor 6, 40 or a contact sensor 30. The sensor signal(s) is/are supplied to the microcontroller 5. For evaluating the sensor signal from the pressure sensor 11 or the flow sensor 16 the microcontroller 5 preferably comprises one or more analog-to-digital converter(s). Commercially available microcontrollers often include an analog-to-digital converter together with a preceding multiplexer so that, for example, one out of eight analog inputs is connected to the analog-to-digital converter.

In a simple embodiment the signal supplied by the microphone may be evaluated merely with respect to its amplitude. If the microphone signal, which is rectified and lowpass-filtered with a time constant of 0.3 to 3 s, exceeds a threshold the microcontroller switches on the illumination. The possibly required amplification of the microphone signal, the rectification of the microphone signal and the lowpass-filtering may be performed in an analog circuit or even digitally in the microcontroller 5.

In a slightly more complex embodiment amplitude variations of the microphone signal may be evaluated. To this end the microphone signal is likewise rectified and thereafter lowpass-filtered with a time constant of approximately 0.1 s. This amplitude signal can, again, be lowpass-filtered with a longer time constant of, for example, 10 s, so as to obtain a mean amplitude signal. On the other hand, the constant component can be filtered out of the amplitude signal, for example, by means of a capacitor, and the alternating component of the amplitude signal can be rectified and again lowpass-filtered, for example, with a time constant of 1 s, so as to obtain an amplitude variation signal. If the amplitude variation signal divided by the mean amplitude value exceeds a threshold the microcontroller 5 switches on the illumination.

If the respiratory apparatus is equipped with an efficient microcontroller, the microcontroller may perform a speech recognition so that the respiratory apparatus is entirely controllable by speech. The speech recognition needs not to be very sophisticated since it only has to understand a few commands like pressure on, pressure off, illumination on, possibly increase pressure, decrease pressure etc.

An accelerometer 21 may serve to detect the slight concussions caused by the patient groping for the CPAP-apparatus and thereby slightly hitting on the CPAP-apparatus. For allowing the optimum detection of such slight impacts the accelerometer 21 is advantageously disposed away from the footprint or foot nipples of the CPAP-apparatus. If the CPAP-apparatus stands on a shelf which is not too stable, the accelerometer 21 can also detect slight concussions of the shelf bottom caused by the groping and subsequently switch on the illumination.

For detecting movements in the proximity of the CPAP-apparatus, e.g. the gripping or groping of the patient for the CPAP-apparatus, an infrared sensor may be applied as an inexpensive solution. This infrared sensor may either be a photodiode 6 or an infrared sensor with a thermopile 40. The photodiode 6 is biased in the non-conducting direction. The more light falls onto the photodiode, the higher is the current through the photodiode. This current is converted into a voltage, for example, at the resistor 7. As the photodiode is to detect movements, the alternating component of the voltage dropping at resistor 7 is advantageously decoupled from the voltage by capacitor 10. The alternating signal supplied by capacitor 10 is furnished to component 12. Component 12 may be a band-limiting amplifier or a rectifying amplifier with subsequent lowpass as to disburden the microcontroller 5. In another embodiment component 12 may be a comparator which already supplies a digital output signal to the microcontroller 5, in particular to an interrupt input of the microcontroller 5.

One possible circuitry of a contact sensor is shown in FIG. 2. The resistor 31 applies a voltage Uₛ to the electrode 32 wherein, in the embodiment described first, the voltage Uₛ is to be a direct voltage. The capacitor 34 decouples the alternating component of the voltage applied to electrode 32 and furnishes said alternating component to component 35. Component 35 in its simplest form may be a comparator which outputs, for example, a logic zero corresponding to 0 V if a voltage underneath a threshold is applied to its input, and otherwise outputs a logic one corresponding, for example, to 5 V. If an operator now contacts the electrodes 32 and 33 simultaneously, the operator constitutes an electric resistance between said two electrodes due to which the voltage at electrode 32 drops. By this a negative voltage pulse is formed by capacitor 34, which may be detected in component 35 and converted into a digital negative pulse. In particular if Uₛ is a direct voltage the capacitor 34 may be omitted.

In another embodiment the voltage Uₛ may be an alternating voltage. In the case of alternating current, too, the amplitude of the alternating voltage at electrode 32 drops if the electrodes 32 and 33 are contacted simultaneously. This can, for example, be detected by a simple comparator, which outputs a square wave alternating voltage signal if the electrodes 32 and 33 are not contacted, and which outputs a direct voltage signal if the electrodes 32 and 33 are contacted. In another embodiment the alternating voltage supplied by the capacitor 34 may at first be rectified, and the amplitude of the rectified alternating voltage may be compared with a threshold in component 35. The contact 36 symbolizes the connection to the microcontroller 5.

In the case of alternating voltage the electrode 33 may be omitted. If a patient contacts the electrode 32 his body acts as a large capacitor against ground as a result of which also the amplitude of the alternating voltage at electrode 32 drops. In this embodiment the electrode 32 may reticulate over the housing of the respiratory apparatus so that the contact sensor 30 detects each movement of the housing. In another embodiment the housing may also be made of a conductive synthetic material which then forms the electrode 32. The conductivity of the synthetic material need not be high as the resistance of the patient's skin also is in the 100 kOhm range.

Infrared sensors, in particular for distant infrared, frequently comprise thermopiles. Said thermopiles measure the temperature difference between a hotter spot 41 and a colder spot 42. The hotter spot 41 is blackened such that it absorbs light in a frequency range as broad as possible, i.e. it appears as black. The thermopile consists of a meander-shaped arrangement of two different conductors which in particular have a different position in the thermoelectric contact series. The thermopile supplies a voltage being proportional by approximation to the incident luminous power. As movements are to be detected, the alternating component of the voltage is preferably decoupled by capacitor 45, amplified by amplifier 46 and furnished to the microcontroller 5 via the terminal 47.

The amplifier 46 may also be replaced by component 12.

It has moreover been found that an infrared sensor also outputs a sensor signal in response to slight concussions of the housing, provided that it is installed according to FIG. 4. FIG. 4 shows an infrared sensor 51, a Plexiglas window 52, housing parts 53, a sensor socket 54, connecting leads 55, a printed circuit board 56 and electrical and electronic components 57. It is essential that the sensor is pressed by its connecting leads with its window against the Plexiglas window 52. To this end it is not necessary that the connecting leads be bent as is shown in FIG. 4. However, in order to also guarantee a reproducible mechanical contact between the infrared sensor 51 and the Plexiglas window 52 during the production, it is advantageous if the connecting leads are bent so as to act as springs.

In the foregoing, the invention was explained in more detail by means of preferred embodiments. It is, however, obvious for a person skilled in the art that various alterations and modifications may be made without departing from the spirit of the invention. Therefore, the scope of protection is defined by the following claims and their equivalents.

### LIST REFERENCE NUMERALS

1 CPAP-apparatus
2 hole
4 housing
5 microcontroller
6 photodiode
7 resistor
8 turbine
9 respiratory hose
10 capacitor
11 pressure sensor
12 component
13 filament bulb
14 resistor
15 light emitting diode
16 flow sensor
17 heating filament
18 respiratory mask
19 sleeping person
20 microphone
21 accelerometer
22 lens
23 LCD
24 operating member
30 contact sensor
31 resistor
32, 33 electrode
34 capacitor
35 component
36 connecting line
40 thermopile
41 hot end
42 cold end
43 first conductor
44 second conductor
45 capacitor
46 amplifier
47 connecting line
51 infrared sensor
52 plexiglas window
53 part of the housing
54 sensor socket
55 connecting leads
56 printed circuit board
57 electric and electronic components

1. A respiratory apparatus, comprising:
an illumination device;
a controller connected with the illumination device for switching the illumination on and off; and
a sensor connected with the controller and outputting a signal to the controller which causes the controller to switch on the illumination.

2. The respiratory apparatus according to claim 1, wherein the controller switches off the illumination if the controller does not receive a signal for a predetermined time causing the controller to switch on the illumination.

3. The respiratory apparatus according to claim 1, wherein the sensor is a microphone.

4. The respiratory apparatus according to claim 3, wherein the controller evaluates the height of the signal supplied by the microphone and switches on the illumination if the amplitude exceeds the predetermined threshold.

5. The respiratory apparatus according to claim 3, wherein the controller evaluates the amplitude of the signal supplied by the microphone and wherein the controller switches on the illumination if the ratio of variations of the amplitude divided by a mean-time value of the amplitude exceeds a threshold.

6. The respiratory apparatus according to claim 1, wherein the sensor is an accelerometer, the accelerometer being disposed away from the footprint of the respiratory apparatus so as to detect accelerations of the respiratory apparatus caused by groping for the respiratory apparatus, the controller switching on the illumination if the signal supplied by the sensor indicates accelerations above a threshold.

7. The respiratory apparatus according to claim 1, wherein the sensor is a movement sensor detecting the movement of objects in the proximity of the respiratory apparatus.

8. The respiratory apparatus according to claim 7, wherein the movement sensor is an infrared sensor the.

9. The respiratory apparatus according to claim 8, wherein the infrared sensor is installed such that it presses against a transparent part of the housing.

10. The respiratory apparatus according to claim 8, wherein the respiratory apparatus moreover comprises optics so as to focus light from a defined solid angle range onto the infrared sensor.

11. The respiratory apparatus according to claim 1, wherein the respiratory apparatus is a CPAP-apparatus.

12. The respiratory apparatus according to claim 1, wherein the illumination device illuminates a display.

13. The respiratory apparatus according to claim 1, wherein the illumination device illuminates operating members.

14. A method for a respiratory apparatus, comprising:
outputting a sensor signal from a sensor of the respiratory apparatus to a controller of the respiratory apparatus which causes the controller to switch on the illumination; and
illuminating a light source in response to the sensor signal.

15. The method according to claim 14, wherein sound is detected by the sensor which is a microphone.

16. The method according to claim 15, wherein the controller evaluates the amplitude of the sensor signal and wherein the controller switches on the illumination if the ratio of variations of the amplitude divided by a mean-time value of the amplitude exceeds a threshold.

17. The method according to claim 14, wherein the sensor detects accelerations of the respiratory apparatus caused by groping for the respiratory apparatus and switching on the illumination if the signal supplied by the sensor indicates accelerations above a threshold.

18. The method according to claim 14, wherein the sensor is a movement sensor detecting the movement of objects in the proximity of the respiratory apparatus.

19. The method according to claim 18, wherein accelerations of the respiratory apparatus are conveyed to an movement sensor by pressing the movement sensor against a transparent part of the housing.

20. The method according to claim 18, wherein the method further comprises focussing light from a defined solid angle range onto the movement sensor.
